(19) United States
(12) Patent Application Publication
(10) Pub. No.: US 2022/0288059 A1
(43) Pub. Date: Sep. 15, 2022

(54) NANOENCAPSULATED CANNABIDIOL

(71) Applicant: Trevor P. CASTOR, Woburn, MA (US)

(72) Inventor: Trevor Percival Castor, Arlington, MA (US)

(73) Assignee: Aphios Corporation, Woburn, MA (US)

(21) Appl. No.: 17/635,694

(22) PCT Filed: Aug. 15, 2020
(86) PCT No.: PCT/US2020/046582

§ 371 Date: Feb. 15, 2022

(51) Int. Cl. A61K 31/485; A61K 9/51; A61K 31/05; A61K 9/19

(52) U.S. Cl. CPC A61K 31/485; A61K 9/5153; A61K 9/5192; A61K 31/05; A61K 9/19

(57) ABSTRACT

Embodiments of the present invention are directed to articles of manufacture and methods of making such articles having utility for the delivery of cannabinoids, naltrexone and a combination of cannabinoids and naltrexone as a therapeutic. One embodiment of the present invention directed to the article of manufacture comprises a lyophilized sphere having a diameter of about 100 to 500 nanometers having a shell comprising a biodegradable polymer containing a cannabinoid, naltrexone and a combination of a cannabinoid and naltrexone in a deareated buffer. A featured cannabinoid is cannabidiol (CBD).

### RELATED APPLICATIONS

This application is related in part of U.S. Pat. No. 8,629,177 issued on Jan. 14, 2014, which is incorporated by reference herein in its entirety.

### GOVERNMENT SUPPORT

Research leading to this invention was in part funded with Grant No. 5R44DA038932-03 from the National Institute on Drug Abuse, United States National Institutes of Health, Bethesda, Md.

### FIELD OF THE INVENTION

The present invention pertains to a product consisting of cannabidiol (CBD), naltrexone and a combination of CBD and naltrexone encapsulated in biodegradable polymer nanospheres as well as a method of encapsulating CBD, naltrexone and a combination of CBD and naltrexone in biodegradable polymer nanospheres and an apparatus for encapsulating CBD, naltrexone and a combination of CBD and naltrexone in biodegradable polymer nanospheres.

### BACKGROUND OF THE INVENTION

The United States consumes 80% of the world supply of prescription opioid analgesics, and opioid prescriptions have climbed by 300% since 1991. In 2017, more than 47,000 Americans died as a result of an opioid overdose, including prescription opioids, heroin, and illicitly manufactured fentanyl, a powerful synthetic opioid. That same year, an estimated 1.7 million people in the United States suffered from substance use disorders related to prescription opioid pain relievers, and 652,000 suffered from a heroin use disorder (not mutually exclusive).

Since 2000 the CDC reported that the US rate of drug overdose mortality increased by 137%, with a 200% increase in the death rate from opioid pain relievers and heroin, between 2000-2014. This increase parallels the vast increase in heroin use across the country and is closely tied to opioid pain reliever misuse and dependence. To fill the gap between current Opioid Use Disorder (OUD) treatments and the widespread prevalence of misuse, relapse, and overdose, the development of novel, alternative, or adjunct treatment OUD therapies is highly warranted.

Unfortunately, few effective treatments are available for individuals trying to recover from opioid addiction and drug dependence, which include methadone, buprenorphine and naltrexone. Methadone and buprenorphine are both opioids themselves, and although effective at detoxing patients and counteracting some of the serious withdrawal side effects of opioid abuse, they do not decrease the more prevalent drug abuse side effects like cravings and relapse. “Craving” is defined as the subjective urge to use substances and is a major motivation for relapse across all drugs deemed addictive.

Naltrexone, an opioid receptor agonist, is approved by the FDA to prevent opioid use relapse in people with no physical dependence to opioid but initiation and adherence to treatment are low. Currently, there is only one FDA approved drug for treating opioid withdrawal, Lofexidine hydrochloride, an oral selective alpha 2-adrenergic receptor agonist that reduces the release of norepinephrine. Buprenorphine treatment, given its partial agonist effects, may offer a unique pharmacological opportunity to reduce the lethality of opioid overdose. Relapse prevention with the opioid agonist naltrexone may also reduce the risk of overdose. These drugs are “flawed” by poor bioavailability and pharmacokinetics that further limit their effective application. Innovative and novel administration formulations are needed to improve the effectiveness and/or minimize the abuse/addictive potential for therapeutic agents used in the treatment of drug abuse/dependence. Ideally, these novel therapies should minimize both craving and abuse potential, without the side effects and adverse reactions that are experienced with current treatment options. This in turn lowers the likelihood of relapse, improve adherence to treatment and sustain recovery.

Low-dose naltrexone (LDN) has been demonstrated to reduce symptom severity in conditions such as fibromyalgia, Crohn's disease, multiple sclerosis, and complex regional pain syndrome. LDN may operate as a novel anti-inflammatory agent in the central nervous system, via action on microglial cells. These effects may be unique to low dosages of naltrexone and appear to be entirely independent from naltrexone's better-known activity on opioid receptors. As a daily oral therapy, LDN is inexpensive and well-tolerated.

Cannabidiol (CBD), a unique, bioactive component of marijuana (Cannabis sativa) is non-psychotropic, non-psychotomimetic and anxiolytic. Studies indicate that CBD acts as an activator of 5-HT_{1A} receptors, which lowers extracellular concentrations of serotonin, potentially eliminating “euphoric” effects of abused opioids. Fentanyl, a synthetic opioid with wide therapeutic use and abuse, is a p-opioid receptor that can also act as a 5-HT_{1A} receptor agonist. 5-HT_{1A} receptors have been associated with brain-reward processes and addiction. Consistent with this concept, CBD also intensifies the agonist effects of naloxone.

Studies performed in vivo indicate that CBD showed dose-dependent anti-nociceptive effects on Wistar rats utilizing the tail-flick test. CBD has also been shown to effectively inhibit cue-induced heroin-seeking behaviors in mice. CBD is now in Phase 2 trials in humans to evaluate the acute and short-term effects on cue-induced cravings in heroin dependent humans. In 2017, findings from a self-report study of a group of adult addicts showed that periods of self-reported intentional use of cannabis to control crack-cocaine use was associated with subsequent periods of reduced use. Their work also called for support to further investigate the therapeutic potential of cannabinoids to attenuate craving and other cocaine-cessation symptoms. Studies have also indicated that intermittent marijuana use is associated with improved retention of naltrexone treatment for opiate-dependence, and ultra-low dose naltrexone enhances cannabinoid-induced anti-nociception. Anecdotal evidence also suggests that CBD works in concert with naltrexone for OUD.

Preclinical studies indicate that CBD interferes with the brain-reward mechanisms responsible for the expression of acute reinforcing properties of opioids. These and other studies imply that CBD is a highly valuable tool in the war against addiction both by relieving the adverse symptoms addicts experience as they attempt recovery, i.e. cravings, anxiety and pain, as well as avoiding therapeutics which are themselves opioids. CBD, unlike buprenorphine and methadone, is not an opioid, and as such has not been shown to exhibit some of the adverse effects associated with buprenorphine or methadone. Current oral delivery of CBD demonstrates that a large fraction of the dose is excreted unchanged, indicative of its low bioavailability (˜6%) in humans. This is largely due to its hydrophobic nature, which reflects slow onset, peak spikes and short-term efficacy. CBD is also metabolized extensively by the liver and therefore “first-pass metabolism” contributes to poor bioavailability. CBD thus also suffers from poor pharmacokinetics and pharmacodynamics.

Chemotherapy-induced peripheral neuropathic pain (CIPNP) is a common adverse effect of many anticancer drugs, such as platinum analogs, Taxol®, other taxanes and vinca alkaloids. Opioid based therapies are in place to offset some of the symptoms associated with CIPNP, but have their own host of negative side-effects and in some cases are deemed ineffective. The anti-depressant Cymbalta (Duloxetine), a selective serotonin and norepinephrine reuptake inhibitor (SSNRI), is recommended as a first line agent for the treatment of chemotherapy-induced neuropathy by ASCO. Cymbalta, however, has suicidal and other side effects including drug-drug interactions. There are few non-opioid, direct pharmaceutical interventions for CIPNP even though more than 50% of cancer patients undergoing chemotherapy suffer from CIPNP. Cannabidiol (CBD) has shown that it inhibits paclitaxel-induced pain through the serotonin 5-HT_{1A} receptor and does so without diminishing chemotherapy efficacy.

CBD is a non-psychoactive component of marijuana and has been shown to possess antipsychotic, anxiolytic, anticonvulsant, anti-inflammatory, and anti-emetic properties. CBD shows limited oral bioavailability because of its lipophilicity and extensive first pass metabolism. CBD is also known for its high intra- and inter-subject absorption variability in humans. Recently (June 2018), an oral formulation of CBD in sesame oil (Epidiolex, GW Pharma) was approved by the FDA for Dravet's syndrome, an orphan form of childhood epilepsy. Dosing ranges from 5 mg/kg/day to 20 mg/kg/day; recommended maximum dosing for a 70 kg adult is 1,400 mg/day. This formulation is very similar to the Marinol formulation of Δ9-THC in sesame oil that is also poorly bioavailable, peak release and suffers from extensive first pass metabolism.

A sustained release, stable, nanoformulation of CBD for treating CIPNP that will be protected from first-pass metabolism. Since CBD is highly hydrophobic and susceptible to acid degradation and oxidation, several challenges remain to formulating a stable and bioavailable CBD therapeutic. These challenges can be met by nanoencapsulating CBD in hydrophobic, biodegradable poly-lactic glycolic acid (PLGA) polymers, Eudragit L100 and polycaprolactone (PCL) nanospheres using SuperFluids™ Polymer Nanospheres (SFS-PNS) technologies. Nanoencapsulation protect CBDs in its passage to the stomach and in its high acid gastric environment. The hydrophobic polymer nanospheres assist in the transport of CBD nanoparticles across the stomach wall into the blood stream and protect CBD from first-pass metabolism in the liver. With degradation of the biodegradable polymer shell, CBD is released in a sustained manner consistent with polymer degradation.

CBD has therapeutic applications for opioid, heroin, morphine and cannabis use disorders; chemotherapeutic induced neuropathic peripheral pain, diabetic neuropathy, peripheral and chronic pain; epilepsy and other seizure disorders; multiple sclerosis and dystonia; Parkinson disease; Huntington disease; Alzheimer's disease; Fragile-X syndrome; Crohn's disease; graft-versus-host disease (GVHD); cancer; diabetes; anxiety and social anxiety disorder; bipolar disorder; schizophrenia; smoking cessation; insomnia; inflammation; bacterial and viral diseases. Nanoencapsulated CBD will improve its therapeutic index for these applications.

Therapeutic uses of cannabinoids have been limited because of social and legal concerns and because means for administering cannabinoids has been lacking. Cannabinoids with therapeutic potential are not particularly stable, degrade readily, are subject to first pass metabolism and low oral bioavailability.

### SUMMARY OF INVENTION

Embodiments of the present invention are directed to articles of manufacture, methods for and apparatus of making such articles having utility for the delivery of cannabinoids as a therapeutic. One embodiment of the present invention directed to the article of manufacture comprises a lyophilized sphere having a diameter of about 100 to 500 nanometers having a shell comprising a biodegradable polymer containing a cannabinoid in a deareated buffer that can be lyophilized or reconstituted in an aqueous buffer for delivery as inhalers, capsules, gel caps, tablets, pills, powders, suspensions, implants and transdermal patches. A featured cannabinoid is cannabidiol (CBD).

A featured hydrophobic biodegradable polymer is a polymer of poly (D,L-lactide-coglycolide polymer) having an acronym PLGA. An embodiment features poly (D,L-lactide-coglycolide polymer) present in a ratio of 75:25 to 25:75 lactide to glycolide. Other embodiments feature ratios of 60:40 to 40:60 and about 50:50. Another featured hydrophobic biodegradable polymer is polycaprolactone having an acronym PCL. Another featured hydrophobic biodegradable polymer is Eudragit L100.

As used herein, the term “deareated buffer” refers to an aqueous solution having an oxygen content lower than a commonly found in tap water or water allowed to stand in an open container for a prolonged period. One embodiment of the present article features a buffer comprising one or more sugars. The sugars are present in the buffer in a concentration of 5 to 20 percent. Another embodiment of the present invention features a buffer comprising an alcohol. The alcohol has a concentration ranging from 1 to 50%. The alcohol can be selected from the group comprising methanol, ethanol, and propanol. A preferred alcohol is ethanol.

As used herein, the buffer is understood to lose much if not all of its alcohol and aqueous content during the lyophilization, leaving non-volatile constituents which return to nanoparticle form upon solvation with protection of particle integrity in the sucrose cryoprotectant.

A further embodiment of the present invention features a cross-linking agent. A preferred cross-linking agent is polyvinyl alcohol having an acronym PVA. The concentration of the cross-linking agent is 0.1 to 10% or more preferably about 1%.

One embodiment of the present invention features a plurality of spheres in a quantity to cause a therapeutic effect. For example, without limitation, a plurality of spheres is held in a dosage form. The dosage form is selected from the group comprising inhalers, capsules, gel caps, tablets, pills, powders, suspensions, implants and transdermal patches.

A further embodiment of the present invention is directed to a method of making a lyophilized sphere having a diameter of about 100 to 500 nanometers having a shell comprising a biodegradable polymer containing a cannabinoid in a deareated buffer. The method utilizes near-critical, critical, and supercritical fluids (hereinafter referred to as SuperFluids™ [SFS]) with or without polar cosolvents such as ethanol and acetone. The method comprises the steps of forming a mixture of one or more biodegradable polymers and a cannabinoid in SuperFluids such as carbon dioxide, propane and Freon-22 held under conditions in which the fluids are supercritical, critical or near critical fluid. The mixture is injected in a stream in a deareated solution comprising a cross-linking agent in a buffer to form one of more spheres having a diameter of 100 to 500 nanometers. The one or more spheres are lyophilized to form a lyophilized sphere having a diameter of about 100 to 500 nanometers having a shell comprising a biodegradable polymer containing the cannabinoid.

One embodiment features the hydrophobic biodegradable polymer poly (D,L-lactide-coglycolide polymer). One embodiment features the cannabinoid cannabidiol in a deareated buffer.

Another embodiment features poly (D,L-lactide-coglycolide polymer) present in a ratio of 75:25 to 25:75, or 60:40 to 40:60, or about 50:50.

Another embodiment features the hydrophobic biodegradable polymer polycaprolactone. Another embodiment features the hydrophobic biodegradable polymer Eudragit L100.

One embodiment features a buffer comprising one or more sugars. Preferably, the sugars are present in the buffer in a concentration of 5 to 20 percent. A further embodiment features a buffer comprising an alcohol. Preferably, the alcohol has a concentration ranging from 1 to 50%. A preferred alcohol is ethanol.

One embodiment of the present invention features a cross-linking agent, such as, without limitation, polyvinyl alcohol. The cross-linking agent is present in the buffer or the spheres are placed in a solution containing the cross-linking agent prior to lyophilization.

In still another embodiment, CBD is co-encapsulated with naltrexone in biodegradable polymer nanospheres, which may be incorporated into formulations for different delivery formats including pills. Naltrexone is hydrophobic and soluble in ethanol. Naltrexone in ethanol can be added to the feed stream of CBD in ethanol in the SuperFluids™ process. Co-encapsulation provides a one-pill drug regimen for the treatment of opioid dependence and pain. Also, lyophilized nanoencapsulated CBD can be combined with naltrexone hydrochloride powder in a gelatin capsule.

The present invention is also useful in the treatment of chemotherapy-induced peripheral neuropathy pain (CIPNP), a common adverse effect of many anticancer drugs, such as platinum analogs, Taxol®, other taxanes and vinca alkaloids. CIPNP impacts about 50% of cancer patients undergoing chemotherapy and there are no effective non-opioid treatments available. Therapies based on opioids are in place to offset some of the symptoms associated with CIPNP, but have their own host of negative side-effects and in some cases are deemed ineffective. Hence, there is a need for a non-opioid, preferably oral, safe and effective formulation to eliminate or control CIPNP.

Cannabidiol (CBD) inhibits paclitaxel-induced pain through the serotonin 5-HT_{1A} receptors without diminishing chemotherapy efficacy. However, CBD is poorly bioavailable because of its hydrophobicity, and thus suffers from relatively poor pharmacodynamics.

A sustained release, non-opioid therapeutic for treating chemotherapy-induced peripheral neuropathic pain (CIPNP) may be provided by nanoformulations of nano-encapsulated CBD using the SFS-created polymer nanospheres of the present invention. In another aspect of the present invention, a sustained release pill formulation uses nanoencapsulation of a CBD-based, non-opioid analgesic that acts on CB1, CB2 and serotonin receptors. A twice-daily pill with low or no addiction potential will impact thousands of patients suffering from CIPNP in the United States and worldwide. Such a pill will also help diabetic patients suffering from diabetes-induced peripheral neuropathy.

The present spheres, having diameters measured in nanometers, sometimes referred to as nanospheres, contain cannabinoids, such as cannabidiol in an environment having limited oxygen and other reactants which degrade the cannabinoid. The articles of manufacture are stable and bioavailable.

These and other features of the present invention will be apparent to those skilled in the art upon viewing the Figures and reading the detailed description which follow.

### BRIEF DESCRIPTION OF THE DRAWINGS

FIG. 1 depicts an article of manufacture having features of the present invention;

FIG. 2 depicts an apparatus for making embodiments of the present invention and performing features of the present method;

FIG. 3 depicts the dilution curve of a cannabidiol standard;

FIG. 4 depicts the HPLC chromatograms of pure CBD and nanoencapsulated CBD; and

FIG. 5 depicts the UV spectra of pure CBD and nanoencapsulated CBD.

### DETAILED DESCRIPTION

Embodiments of the present invention will be discussed in detail as to what the inventor considers to be the best mode, with respect to an article of manufacture comprising a lyophilized sphere having a diameter of about 100 to 500 nanometers having a shell comprising poly (D,L-lactide-coglycolide polymer), polycaprolactone and Eudragit containing cannabidiol in a deareated buffer. Those skilled in the art will readily understand that such embodiments are subject to modification and alteration without departing from the teaching herein. Therefore, the invention should not be limited to these precise details.

As used herein, the term “cannabidiol” or “CBD” is used in the normal chemical sense of the compound C₂₁H₃₀O₂ (MW=314.46). CBD is a non-psychoactive constituent of marijuana. The LD₅₀ in rhesus monkeys (mg/kg) is 212 mg/kg intravenously, the LD₅₀ in dogs (mg/kg) is 254 mg/kg intravenously, LD₅₀ in mice is 50 mg/kg intravenously. CBD is, however, less toxic than Δ9-THC by i.v. studies for LD₅₀ in rats.

CBD and Δ9-THC have identical molecular weights, comparable UV spectra and similar pKa ranges, however CBD may not exhibit characteristics similar to that of the nanoencapsulation of Δ9-THC, since CBD has two free —OH functional groups and is a more flexible structure as compared to —OH in THC having a more rigid tricyclic structure.

As used herein, the terms “critical,” “supercritical” and “near critical” are used in their physical-chemical sense to mean one or more compounds under conditions that are supercritical, critical or near critical. A pure compound enters its supercritical fluid region at conditions that equal or exceed both its critical temperature and critical pressure. These critical parameters are intrinsic thermodynamic properties of all sufficiently stable pure component compounds. Carbon dioxide, for example, becomes supercritical at conditions that equal or exceed its critical temperature of 31.1° C. and its critical pressure of 72.8 atm (1,070 psig). In the supercritical or near-critical fluid region, normally gaseous substances, such as carbon dioxide, become dense phase fluids that have been observed to exhibit greatly enhanced solvating, selection, penetration and expansion power as compared to the gaseous state. At a pressure of 3,000 psig (204 atm) and a temperature of 40° C., carbon dioxide has a density around 0.8 g/cc and behaves very much like a nonpolar organic solvent. The density of supercritical fluid is strongly dependent on both temperature and pressure-temperature changes of tens of degrees or pressure changes by tens of atmospheres can change solubility by an order of magnitude or more.

As used herein, the term “biodegradable” refers to materials that are broken down in the body to nontoxic products (lactic acid and glycolic acid) and have been approved by the FDA for use as resorbable sutures, in bone implants and as controlled release microspheres. The most commonly used biodegradable polymers are of the poly(hydroxyacid) type, in particular poly(L-lactic acid), poly(D,L-lactic acid), poly(glycolic acid) and copolymers thereof.

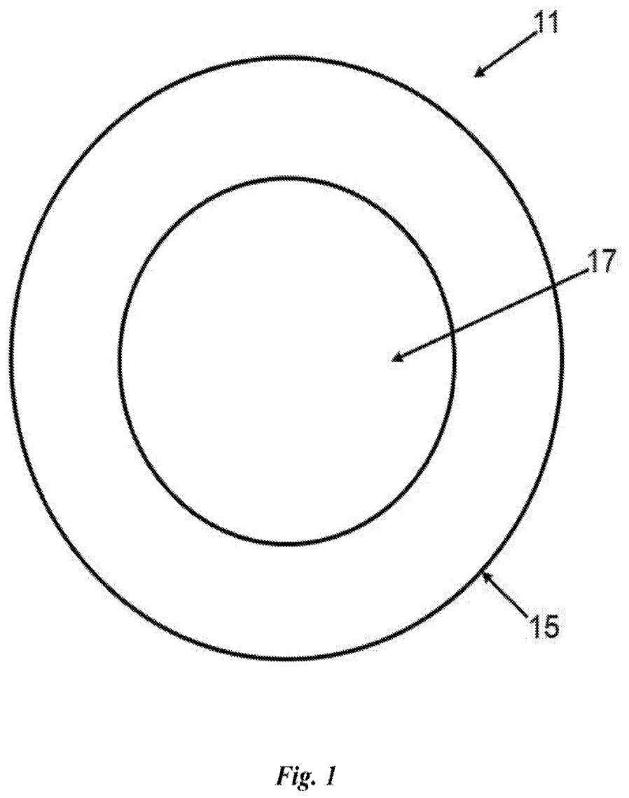

Turning now to FIG. 1, an embodiment of the present invention directed to the article of manufacture, a lyophilized sphere, generally designated by the numeral 11, is depicted in cross-sectional view. The sphere 11 has a diameter of about 100 to 500 nanometers. The sphere 11 has a shell 15 comprising a biodegradable polymer containing a cannabinoid with or without low dose naltrexone in a deareated buffer. The sphere has an interior 17 comprising the biodegradable polymer which could be fully, partially or not cross linked and a cannabinoid with or without low dose naltrexone in a deareated buffer. A featured cannabinoid is cannabidiol (CBD). The shell 15 and the interior 17 differ in that the polymer of the shell is cross-linked. The deareated buffer in this context refers to the non-volatile components of the buffer, for example, one or more sugars which may migrate into the shell 15 and interior 17 upon formation.

The example features a polymer of poly (D,L-lactide-coglycolide polymer), Eudragit L100 and polycaprolactone. Referring now to poly (D,L-lactide-coglycolide polymer), this polymer is present in a ratio of 75:25 to 25:75 lactide to glycolide. Other embodiments feature ratios of 60:40 to 40:60 and about 50:50. Other embodiments include mixtures of the polymers. For example, the poly (D,L-lactide-coglycolide polymer) and polycaprolactone are used in a ratio of about 2 to 1 to 1 to 2 parts by weight lactide-coglycolide to polycaprolactone. These polymers readily form a solution of about one to one part by weight.

The deareated buffer is an aqueous solution having a low oxygen; for example, water which has been held under low pressure in the absence of atmospheric gases. One embodiment of the present article features a buffer comprising one or more sugars. The sugars are present in the buffer in a concentration of 5 to 20 percent. Another embodiment of the present article features a buffer containing pH buffering agents. The pH buffering pH agents are citric acid in a concentration of 0.0033 percent. Another embodiment of the present invention features a buffer comprises an alcohol. The alcohol has a concentration ranging from 1 to 50%. The alcohol can be selected from the group comprising methanol, ethanol, and propanol. A preferred alcohol is ethanol.

Again, the buffer is understood to lose much if not all of its alcohol and aqueous content during lyophilization, leaving non-volatile constituents which return to solution upon solubilization as part of the bio-degradation of the shell and/or the adsorption of water from the environment.

The shell 15 has a cross linking agent. A preferred cross-linking agent is polyvinyl alcohol. The concentration of the linking agent in the buffer or in a shell forming solution is 0.1 to 10% or more preferably about 0.1%.

A plurality of spheres is held in a dosage form [not shown] in a quantity to cause a therapeutic effect. The dosage form is selected from the group comprising inhalers, capsules, gel caps, tablets, pills, powders, suspensions, implants and transdermal patches.

A further embodiment of the present invention is directed to a method of making a lyophilized sphere 11 having a diameter of about 100 to 500 nanometers having a shell 15 comprising a biodegradable polymer containing a cannabinoid in a deareated buffer. The method comprises the steps of forming a mixture of one or more biodegradable polymers and a cannabinoid with or without low dose naltrexone in carbon dioxide held under conditions in which carbon dioxide is a supercritical, critical or near critical fluid. The mixture is injected in a stream in a deareated solution comprising a cross-linking agent in a buffer, to form one of more spheres having a diameter of 100 to 500 nanometers. The one or more spheres are lyophilized to form a lyophilized sphere having a diameter of about 100 to 500 nanometers having a shell comprising a biodegradable polymer containing the cannabinoid in the deareated buffer.

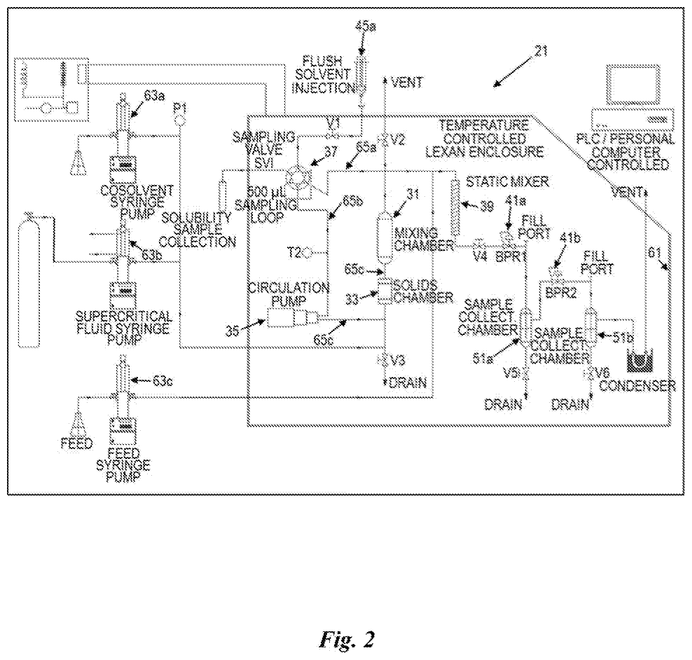

An apparatus, generally designated by the numeral 21, for performing an embodiment of the present invention, is depicted in FIG. 2. The apparatus 21 has the following major components: a mixing chamber 31, a solids chamber 33 for containing the polymer(s), a high pressure circulation pump 35, a multi-port sampling valve 37 (Valco), a static in-line mixer 39, two back pressure regulators 41a and 41b (BPR), two injectors 45a and 45b and two sample collection chambers 51a and 51b all contained in a temperature controlled chamber 61. External to this chamber, three syringe pumps 63a, 63b and 63c (Isco, Inc., Lincoln, Nebr.), are used for delivery of the supercritical fluid, cosolvent and solution of CBD with or without low dose naltrexone. The mixing chamber 31, solids chamber 33, circulation pump 35 and sampling valve 37 are connected in a high-pressure circulation loop represented by conduits 65a, 65b and 65c (and intervening fluid components) with a total volume of approximately 20 ml. The outlets of the supercritical fluid and cosolvent syringe pumps 63a and 63b are connected at a mixing “T” and fed into the high-pressure circulation loop at the entrance of the solids chamber.

There are two take-offs from the high-pressure circulation loop. The first take-off can be achieved by switching the sample valve 37 to allow the circulating stream to flow through a 500 microliters-sampling loop. After the sample is trapped, the sampling loop is flushed with a liquid solvent such as acetone to collect the polymer dissolved in 500 microliters of supercritical, critical or near critical carbon dioxide with or without cosolvents such as an alcohol. The second take-off from the high-pressure circulation loop is at the top of the mixing chamber 31. This take-off is connected to the inlet of static in-line mixer 39. The feed syringe pump for a cannabinoid rich stream is connected to the inlet of the static in-line mixer 39.

In the alternative, a second chamber [not shown] is added to the high-pressure circulation loop to contain cannabinoid with or without low dose naltrexone. Or, as a further alternative, cannabinoid with or without low dose naltrexone is added directly to the polymer in the solids chamber 33. Sample collection chambers 51a and 51b have a 10-mil (internal diameter of 0.25 mm or 250 micron) capillary injection nozzle. Larger internal diameter 316 stainless steel capillary tubes can be used to manufacture larger particle sizes. Impingement nozzles (Bete Fog Nozzle, Inc., Greenfield, Mass.) can also be used. Nozzle impingement will prevent the coagulation of polymeric particles resulting from high concentrations of polymer particles formed under rapid flow conditions.

The apparatus 21 is maintained as a closed system. The entire apparatus up to the backpressure regulators 41a and 41b is designed to operate up to 5,000 psig and 60° C. The apparatus 11 is cleaned in-place by washing with a series of solvents including bleach, caustic, dilute hydrochloric acid, 100% ethanol and then sterilized in-place with an ethanol/water (70/30) mixture.

Biodegradable polymers used in the following examples include pharmaceutical-grade Resomer® RG-502 [poly (D,L-lactide-co-glycolide) 50:50] polymer (Boehringer Ingelheim KG), Eudragit L100 and polycaprolactone (PCL). Specifications are presented in Table 1.

TABLE 1
Specifications of Polymers

| | | | Molecular | Glass |
|---|---|---|---|---|
| | | | Weight | Transition |
| Polymer | Chemical Formula | Polymer Composition | (grams/mole) | Range (° C.) |
| PCL | (C₆H₁₀O₂)ₙ | Polycaprolactone | 14,000 | −60 |
| Endragit L100 | (C₄H₄O₂)ₙ(C₄H₆O₂)ₙ | Poly(methacrylic acid- | 125,000 | >150 |
| | | co-methacrylate) 1:1 | | |
| PLGA | (C₃H₄O₂)ₙ(C₂H₂O₂)ₘ | Poly(D,L-lactide-co- | 7,000-17,000 | 40-55 |
| | | glycolide) 50:50 | | |

Pegylated PLGA impacts both the rate of uptake in the stomach and the circulation time of the nanoencapsulated CBD in the body. Polyethylene glycols (PEGs) are nontoxic and amphophilic, i.e. soluble both in both water and most organic solvents. In pegylation, polyethylene glycols are covalently attached to PLGA, increasing the size of the molecule so it is less likely secreted through the kidney while protecting CBD from degradation. In addition to increasing biological half-life, pegylation improves stability and water solubility, and immunologic characteristics. There may be a trade-down in improving water solubility in that controlled release may be adversely impacted. Specifications of the pegylated PLGA polymers are listed in Table 2.

TABLE 2
Specifications for Pegylated PLGA

| | Inherent | |
|---|---|---|
| | Viscosity | |
| Type | (dL/g) | DL-lactide/glycolide mole ratio |
| RGP t 50106 | Triblock | 10% PEG with 6,000 Dalton |
| RGP d 5055 | Diblock | 5% PEG with 5,000 Dalton |
| RGP d 50105 | Diblock | 10% PEG with 5,000 Dalton |
| RGP d 50155 | Diblock | 15% PEG with 5,000 Dalton |

RG: polyester part (A)-poly(DL-lactide-glycolide) 50:50 ratio
P: PEG (B)

Polymer nanospheres are formed by injecting the polymer-rich, cannabinoid with or without low dose naltrexone laden carbon dioxide fluid with one or more entrainers such as an alcohol into a 0.1% polyvinyl alcohol (PVA) deareated buffer solution. The buffer preferably contains a sugar such as sucrose. Other media can be used, such as high concentration sucrose solutions to aid in particle stability during lyophilization, liquid nitrogen for freezing the particles, and phosphate-buffered saline at physiological pH and citric acid as a control. Other collection media parameters that impact the size and uniformity of the nanospheres are temperature and pressure. Lower temperatures are much more favorable for polymer and CBD stabilities. Operating pressure as well as pressure in the particle formation chamber control the size and uniformity of bubbles formed and nanospheres generated. The pressure in the particle formation chamber can be varied from the vapor pressure of the neat supercritical, critical or near critical fluid at the temperature of the medium to atmospheric pressure.

Optimum polymer nanospheres formation, size and CBD encapsulation depends on the ratio of polymer to CBD in the sample collection chamber(s). This ratio depends on the flowrate of the CBD-rich stream and its concentration, and the flowrate of the polymer-rich supercritical, critical or near critical fluid stream and its concentration (which is defined by polymer solubility at operating conditions). The polymer:CBD ratio can be varied from 100:1 to 1:1. Should there be problematic aggregation of the polymer nanospheres after their formation, the agglomeration is broken by disaggregated utilizing the expansive forces of supercritical fluids, critical fluids or near-critical fluids.

Particle Size: Particle sizes and distributions of the formulations were determined by laser beam interferometer, using a Coulter 4MD submicron particle size analyzer with a range of 30 Angstroms to 3 microns. This technique utilizes photon correlation spectroscopy of the Brownian motion of particles suspended in a liquid to determine the particle size. Multiple-angle detection on the 4MD allows for better characterization of polydisperse samples. These analyses provide: (i) unimodal size analyses that have only mean size and standard deviation; (ii) size distribution analyses that yield information about polydispersity of the sample; and (iii) for the Coulter N4 Plus, “fingerprint,” a procedure that uses the multiple angle measurement provided by the instrument to detect contamination of a sample by particles larger or smaller than the main distribution.

CBD Analytical Method: A modified isocratic method can be utilized with a Phenomenex Luna 5 μm C18 column (15 cm×4.6 mm) with a pre-column at 30° C. The mobile phase, at 2.0 mL/min, consists of 85% acetonitrile in water. Absorbance is monitored by a Waters Photodiode Array (PDA) detector, Model 996, and measured at 285 nm. The HPLC system consists of a Waters Alliance 2695 and is controlled by Waters Empower software. Column temperature is controlled at 30° C. by an Eppendorf CH-30 column heater. HPLC run time is 10 minutes. Analyses are performed in triplicate. Standards are run on the developed HPLC protocol to establish standard regression curves, limits of quantification, and purity of CBD. The purities of the standards and samples are determined using Millennium Software for: (1) Peak Purity Testing which compares all spectra within a peak to the peak apex spectrum to determine if a peak is spectrally homogeneous from liftoff to touchdown; (2) Multicomponent Peak Purity Testing which performs iterative peak purity comparisons to evaluate if there are multiple, spectrally distinct compounds in a peak; and (3) Library Matching which compares an unknown (peak apex) spectrum to known standard spectra in a library to identify a compound.

Dissolution Characteristics: In vitro dissolution were conducted to determine the rate at which the untreated CBD polymer nanospheres dissolve. Ideally, an in vitro dissolution test should reflect the in vivo solubilization conditions. Real in vivo conditions are complex and may include particle-particle interactions that lead to particle aggregation, position dependent permeability and metabolism, changing pH, luminal content and hydrodynamics in the GI tract.

Stability Studies: Shelf stability studies can be conducted with CBD, CBD polymer nanospheres and formulations of the aforementioned. The following tests can be performed: (i) physical appearance; and (ii) CBD content and integrity. Statistical analysis of the data sets can be performed using SYSTAT®.

Encapsulation Efficiency: The loading efficiency of CBD in polymer nanospheres can be determined by dissolving a known amount of nanospheres in a 90% acetonitrile aqueous solution. The amount of CBD was determined by HPLC assay, and the loading efficiency is calculated based on weight percent.

In Vitro Release Studies: In vitro release kinetics of nanospheres were carried out by placing a sample of nanospheres in PBS buffer (pH=7.4), simulated gastric fluid and plasma at 25° C. and 37° C. At intervals of minutes, hours and days, samples were taken and CBD were measured by HPLC in triplicate. Relating the amount of CBD in the supernatant to the total amount in the sample of nanospheres allows determination of cumulative CBD released as a function of time.

### EXAMPLES

### Example 1: CBD Qualification Experiments

### Materials and Equipment:

Waters 717 Autosampler
Waters 996 Photodiode array detector (PDA)
Waters 600 Controller
Waters Model 600 Pumps running on Empower 2, Release 5
Wheaton 1 Liter Glass Bottles
Phenomenex Luna C18(2) 250×4.6 mm 5 μm 100 Å column (SN: H17-396024)
0.22-micron Pre-Column Filter; Filter Insert Assembly (Part #WAT084560) with Inline Filter (Part #WAT035190)
Guard Column (Part #AJ0-4287)

### Standard:

| | | | Calc. Conc. | | | |
|---|---|---|---|---|---|---|
| | Grav. Conc. | | (Grav. Conc. * | | Catalog | Lot |
| Standard | (mg/mL) | Purity | Purity) (mg/mL) | Manufacturer | Number | Number |
| Cannabidiol | 1000 | 99% | 990* | Restek | 34011 | A0124611 |
| (CBD) | | | | | | |

### Solvents:

HPLC grade Methanol (MeOH)
HPLC grade Acetonitrile (ACN)
Glacial Acetic Acid (100%)
Deionized (DI) water
Helium gas

### Supplies:

Hamilton gas tight syringes, various volumes
Waters 150 μL HPLC vial insert
1 mL Amber vials

### Isocratic Solvent Systems:

80% ACNA (800 mL Acetonitrile, 1 mL Acetic Acid, 199 mL deionized water). The mobile phase is degassed by 10 minutes of sonication and 6 minutes under vacuum before use, and sparged with helium gas at 100 psig for 12 seconds of every minute during usage.
85% ACNA (850 mL Acetonitrile, 1 mL Acetic Acid, 149 mL deionized water). The mobile phase is degassed by 10 minutes of sonication and 6 minutes under vacuum before use, and sparged with helium gas at 100 psig for 12 seconds of every minute during usage.

Experiments performed on the HPLC equipment follow the latest revised (07/29/16) standard operating procedure APH-EQ-016, HPLC System, Waters 600 Controller and 717 Autosampler with 996 PDA.
1. Specificity: Specificity is determined by comparing the UV spectra of the eluted compound with data from literature reference stated, looking for quantitative reduction in peak area and differentiation from the solvent, and observing that the retention times differ between the standards.
   Prepare set of 3 samples of each standard.
      100 μL of 990 μg/mL CBD standard=990 μg/mL
      100 μL pure MeOH=0 μg/mL
      50 μL of 990 μg/mL CBD standard, 50 μL MeOH=495 μg/mL
   Allow HPLC system to equilibrate with isocratic ACNA mobile phase at 2 mL/minute for 60 minutes
   Prior to injection of sample set, the injector is purged following standard conditions.
   Inject samples three times under following column conditions:
      Column: Phenomenex 5 μm C18(2)
      Temperature: 30° C.
      Mobile Phase: 80% ACN, 19.9% DI water, 0.10% Acetic Acid
      Wavelength: 285 nm
      Flow Rate: 2 mL/minute
      Injection Volume: 10 μL
      Run time: 20 min/injection
2. Linearity: A standard dilution curve is produced to show linearity of the method.
   According to The International Council for Harmonization (ICH) of Technical Requirements for Pharmaceuticals for Human Use standard, a minimum of five concentrations is recommended. Standard dilution curves are produced for the CBD standard using the following concentrations: 990 μg/mL, 792 μg/mL, 594 μg/mL, 396 μg/mL, and 198 μg/mL. All injections are performed in triplicate using the isocratic solvent system 80% ACNA and previously stated column conditions.
   Linearity is assessed by analyzing the data on an Excel spreadsheet to determine the equation of the straight line and its standard deviation.
3. Range:
   The range is determined by analyzing the data from the Linearity experiment and finding the interval between the highest and lowest values that are in a straight line.
4. Limit of Detection (LOD):
   These studies are performed with starting concentrations of 2.0 μg/mL of the CBDA and CBN standards. 20.0 μg/mL starting concentrations are used for Δ9-THC, Δ9-THCA and CBD standards.
   Each standard is serially diluted (1:2) until the LOD is achieved.
   The LOD is determined by using the concentration at which the average peak height is 3 times the baseline noise. The concentration of each analyte is confirmed by calculating the concentration based on the average peak area and using the slope of the line from the standard dilution curves.
   The initial test concentration and estimated run times for determining LOD and LOQ are listed in Table 3.

TABLE 3
LOD and LOQ Initial Test Concentrations
and Run Times for Cannabidiol Standard

| Sample | Concentration (μg/mL) | | | Run Time |
|---|---|---|---|---|
| name | Injection 1 | Injection 2 | Injection 3 | (min) |
| CBD | 20.0 | 2.0 | 0.2 | 20 |

5. Limit of Quantitation (LOQ):
   The LOQ is determined by using the concentration at which the peak height is 10 times the baseline noise. The concentration of each analyte is confirmed by calculating the concentration based on the average peak area and using the slope of the line from the standard dilution curves. Injections are repeated 3 times.
6. Accuracy: Accuracy is analyzed by comparing the closeness of the test results to true value. It is quantified by determining the standard deviation and % relative standard deviation (% RSD).
   Three samples of CBD standard are prepared at concentrations of 99 μg/mL, 49.5 μg/mL and 24.75 μg/mL. The total volume each sample is 100 μL.
   Each sample is injected three times. The isocratic solvent system 80% ACNA is used for the mobile phase.
   The concentration of each is calculated utilizing the slope of the standard dilution curve.
7. Robustness: By recreating our specificity experiment with a parameter adjustment of an isocratic solvent system of 85% ACNA instead of 80% ACNA, we can compare the data and show the reliability or robustness of our methods.
   Prepare set of 3 samples of each standard.
      100 μL of 99 μg/mL CBD standard=99 μg/mL
      100 μL pure MeOH=0 μg/mL
      50 μL of 99 μg/mL CBD standard, 50 μL MeOH=49.5 μg/mL
   Allow system to equilibrate with isocratic 85% ACNA mobile phase at 2 mL/minute for 60 minutes.
   Prior to injection of sample set, the injector is purged following standard conditions.
   Inject samples three times under following column conditions:
      Column: Phenomenex 5 μm C18(2)
      Temperature: 30° C.
      Mobile Phase: 85% ACN, 29.9% DI water. 0.1% Acetic Acid
      Wavelength: 285 nm
      Flow Rate: 2 mL/minute
      Injection Volume: 10 μL
      Run time: 20 min/injection

Starting with the specificity experiment, three samples were prepared and three injections were performed on each. The samples were made as follows, 100 μL of 99 μg/mL CBD standard, 100 μL pure MeOH, and 50 μL of 99 μg/mL CBD standard, 50 μL MeOH. The results of the specificity experiment for CBD are listed in Table 4.

TABLE 4
Specificity Results for Cannabidiol Standard

| | | | | Retention | Average of | | |
|---|---|---|---|---|---|---|---|
| Conc. | Peak Area in AU | | | Time | Peak Areas | Std. | RSD |
| (μg/mL) | Inj. 1 | Inj. 2 | Inj. 3 | (min) | (AU) | Dev. | (%) |
| 99.0 | 44,932 | 43,956 | 43,429 | 5.1 | 44,106 | 763 | 1.73 |
| 0.0 | 0 | 0 | 0 | 0.0 | 0 | 0 | 0.00 |
| 49.5 | 21,369 | 21,144 | 21,227 | 5.1 | 21,247 | 114 | 0.54 |

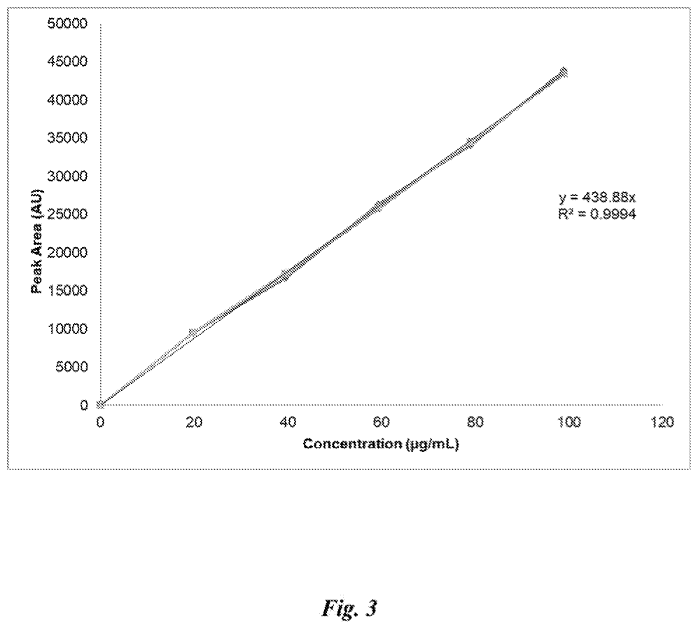

A standard dilution curve was produced to show linearity of the method using concentrations of 99 μg/mL, 79.2 μg/mL, 59.4 μg/mL, 39.6 μg/mL and 19.8 μg/mL. All injections were performed in triplicate using the isocratic solvent system 80% ACNA and previously stated column conditions. Linearity was then assessed by analyzing the data on an Excel spreadsheet to determine the equation of the straight line, the R² value and its standard deviation. Results of the linearity experiment for CBD are listed in Table 5, and the standard linear curve of CBD is shown in FIG. 3. The linear range of the CBD standard curve is between 20-99 μg/mL,

TABLE 5
CBD Standard curve Measured at 285 nm

| Concentration | Peak Area in AU | | | Average of Peak | Standard | RSD |
|---|---|---|---|---|---|---|
| (μg/mL) | Inj. 1 | Inj. 2 | Inj. 3 | Areas (AU) | Deviation | (%) |
| 0 | 0 | 0 | 0 | 0 | 0 | 0 |
| 19.8 | 9,518 | 9,495 | 9,574 | 9,529 | 40.6 | 0.43 |
| 39.6 | 16,610 | 17,703 | 17,360 | 17,058 | 395.6 | 2.31 |
| 59.4 | 26,373 | 75,758 | 26,121 | 26,084 | 309.2 | 1.19 |
| 79.2 | 34,058 | 34,514 | 34,562 | 34,378 | 278.2 | 0.81 |
| 99.0 | 43,908 | 43,385 | 43,407 | 43,567 | 295.8 | 0.68 |

Injection accuracy was performed as required by ICH standards by acquiring at least three injections of three different concentrations. Three samples of CBD were prepared as follows, using 100 μL of 198 μg/mL CBD, 50 μL of 198 μg/mL CBD with 50 μL of 100% MeOH, and 25 μL of 198 μg/mL CBD with 75 μL of 100% MeOH. Samples were then injected three times, using the isocratic solvent system 80% ACNA as the mobile phase. The results on injection accuracy experiments of CBD are listed in Table 6.

TABLE 6
Accuracy Results for Cannabidiol Standard

| Concentration | Peak Area | Calculated Conc. | Difference | RSD |
|---|---|---|---|---|
| (μg/mL) | (AU) | (μg/mL) | (%) | (%) |
| 198.0 | 90,410 | 206.00 | −4.04 | 2.80 |
| 198.0 | 90,671 | 206.60 | −4.34 | 3.00 |
| 198.0 | 90,315 | 205.79 | −3.93 | 2.73 |
| 99.0 | 44,098 | 100.48 | −1.49 | 1.05 |
| 99.0 | 44,046 | 100.36 | −1.37 | 0.96 |
| 99.0 | 44,393 | 101.15 | −2.17 | 1.52 |
| 49.5 | 21,784 | 49.64 | −0.27 | 0.19 |
| 49.5 | 21,703 | 49.45 | 0.10 | 0.07 |
| 49.5 | 21,798 | 49.67 | −0.34 | 0.24 |

Limit of Detection (LOD) and Limit of Quantification (LOQ) studies were performed with starting concentrations based on initial concentrations and run times; results are listed in Table 3. Once these baseline concentrations were identified, the compound was diluted until LOD was established. LOQ was determined by using the concentration at which the peak height was 10 times the baseline noise. The concentration of CBD was confirmed by calculating the concentration based on the peak area and using the slope of the line from the standard dilution curves. Once the appropriate concentrations for LOD and LOQ were found, the injection was repeated 3 times for LOD and 3 times for LOQ. The LOD and LOQ experimental results for CBD are listed in Table 7.

TABLE 7
LOD & LOQ Experimental Results for Cannabidiol Standard

| | Slope of Standard Curve | LOD | LOQ |
|---|---|---|---|
| Standard | (AU/(μg/mL)) | (μg/mL) | (μg/mL) |
| CBD | 438.88 | 0.35 | 20.0 |

The robustness experiment was the specificity experiment recreated with a parameter adjustment, an isocratic solvent system of 85% ACNA instead of 80% ACNA, so that the data can be compared to show the reliability or robustness of the methods. Results of the robustness experiment of CBD are listed in Table 8.

TABLE 8
Robustness and Specificity Comparison for Cannabidiol Standard

| | | | | | | | Isocratic |
|---|---|---|---|---|---|---|---|
| | | | | Average | Average | | Solvent |
| Conc. | Peak Area in AU | | | Retention | Peak Areas | | System |
| (μg/mL) | Inj. 1 | Inj. 2 | Inj. 3 | Time (min) | (AU) | Std. Dev. | Used |
| 99.0 | 44,932 | 43,956 | 43,429 | 5.1 | 44,106 | 763 | 80% |
| | | | | | | | ACNA |
| 0 | 0 | 0 | 0 | 0.0 | 0 | 0 | 80% |
| | | | | | | | ACNA |
| 49.5 | 21,369 | 21,144 | 21,227 | 5.1 | 21,247 | 114 | 80% |
| | | | | | | | ACNA |
| 99.0 | 44,665 | 44,677 | 44,575 | 3.7 | 44,639 | 55.75 | 85% |
| | | | | | | | ACNA |
| 0.0 | 0 | 0 | 0 | 0.0 | 0 | 0 | 85% |
| | | | | | | | ACNA |
| 49.5 | 22,230 | 21,484 | 21,856 | 3.8 | 21,857 | 373 | 85% |
| | | | | | | | ACNA |

### Example 2: CBD Nanoencapsulation in PLGA Polymer with Superfluids Carbon Dioxide and Acetone Cosolvent

CBD was encapsulated in PLGA polymer nanospheres using SFS carbon dioxide and acetone cosolvents with different concentrations of cosolvents. In each experiment, the polymer and CBD enriched SuperFluids stream were decompressed into a 0.1% PVA solution.

CBD-II-114: CBD nanoencapsulation using 50:50::PLGA polymer with P:D molar ratio of 4 and mass ratio of 2 using SFS CO2:Acetone::95:5 at P=2,500 psig and T=45 C and 0.01″ injector into 10.0% sucrose buffer containing 0.1% PVA, 0.033% citric acid and 40% ethanol and pH=4.0.

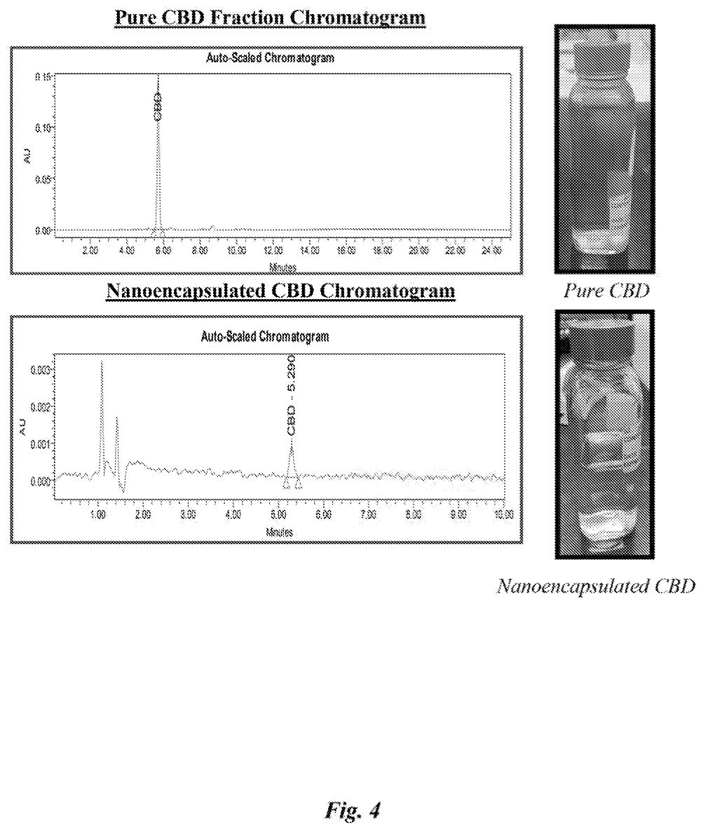

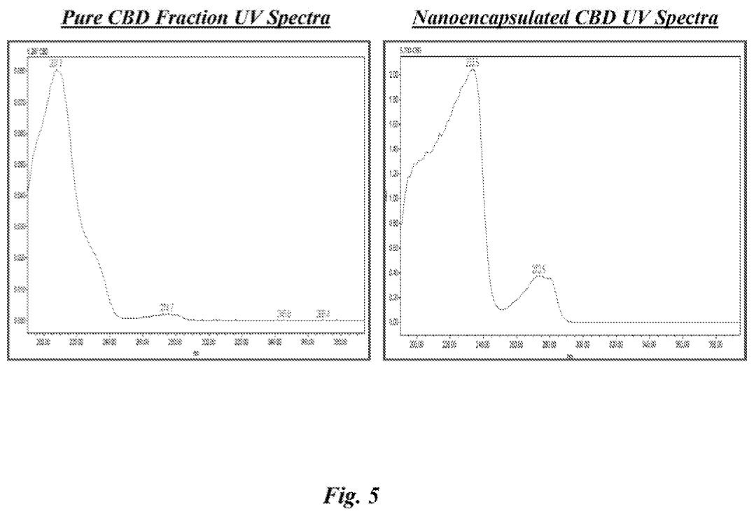

The chromatograms and UV spectra of pure CBD and nanoencapsulated CBD are shown in FIGS. 4 and 5 respectively.

### Example 3: CBD Nanoencapsulation in Eudragit L100 Polymer with Supercritical Carbon Dioxide

CBD was encapsulated in Eudragit L100 polymer nanospheres using SFS carbon dioxide. In each experiment, the polymer and CBD enriched SuperFluids stream were decompressed into a 0.1% PVA solution.

CBD-II-125: CBD was nanoencapsulated using Eudragit L100 polymer with P:D molar ratio of 0.03 and mass ratio of 10 using SFS CO2 at P=1,600 psig and T=45° C. and 0.01″ injector into 10.0% sucrose buffer containing 0.1% PVA, 0.033% citric acid and 40% ethanol and pH=4.0.

### Example 4: CBD Nanoencapsulation in Polycaprolactone Polymer with Supercritical Carbon Dioxide

CBD was encapsulated in polycaprolactone (PCL) polymer nanospheres using SFS carbon dioxide. In each experiment, the polymer and CBD enriched SuperFluids stream were decompressed into a 0.1% PVA solution.

CBD-II-123: CBD was nanoencapsulated using polycaprolactone (PCL) L100 polymer with a P:D molar ratio of 0.24 and a mass ratio of 10 using SFS CO2 at P=3,000 psig and T=50° C. and 0.01″ injector into 10.0% sucrose buffer containing 0.1% PVA, 0.033% citric acid and 40% ethanol and pH=4.0.

### Example 5: CBD Nanoencapsulation in Eudragit L100 and Polycaprolactone Polymer with Supercritical Carbon Dioxide

CBD was encapsulated in Eudragit L100 and polycaprolactone (PCL) polymer nanospheres using SFS carbon dioxide. In each experiment, the polymer and CBD enriched SuperFluids stream were decompressed into a 0.1% PVA solution.

CBD-II-124: CBD was nanoencapsulated using polycaprolactone (PCL) L100 polymer with a P:D molar ratio of 0.24 and a mass ratio of 10 using SFS CO2 at P=3,000 psig and T=50° C. and 0.01″ injector into 10.0% sucrose buffer containing 0.1% PVA, 0.033% citric acid and 40% ethanol and pH=4.0.

While this invention has been particularly shown and described with references to specific embodiments, it will be understood by those skilled in the art that various changes in form and details may be made therein without departing from the spirit and scope of the invention as defined by the appended claims.

What is claimed is:

1-30. (canceled)

31. An article of manufacture comprising a lyophilized sphere having a diameter of about 100 to 500 nanometers having a shell comprising hydrophobic polymers containing cannabinoid and naltrexone.

32. The article of manufacture of claim 31 wherein said hydrophobic polymer is poly (D,L-lactide-coglycolide) wherein the poly (D,L-lactide-coglycolide) is present in a ratio of 75:25 to 25:75.

33-34. (canceled)

35. The article of manufacture of claim 32 wherein the poly (D,L-lactide-coglycolide) polymer is pegylated with polyethylene glycol.

36. The article of manufacture of claim 31 wherein said hydrophobic polymer is selected from the group consisting of polycaprolactone and Eudragit L100.

37. The article of manufacture of claim 31 wherein said hydrophobic polymer is a mixture selected from the group consisting of poly (D,L-lactide-coglycolide), polycaprolactone, and Eudragit L100.

38-43. (canceled)

44. The article of claim 31 comprising a plurality of spheres in a quantity to cause a therapeutic effect.

45. The article of claim 44 wherein said plurality of spheres is held in a dosage form which is selected from the group comprising inhalers, capsules, gel caps, tablets, pills, powders, suspensions and transdermal patches.

46. A method of making a lyophilized sphere having a diameter of about 100 to 500 nanometers having a shell comprising of a hydrophobic polymer or a mixture of hydrophobic polymers containing cannabidiol and naltrexone, said method comprising the steps of:
(a) forming a mixture of hydrophobic polymers, cannabidiol and naltrexone in a supercritical, critical or near-critical fluid such as carbon dioxide, propane or Freon-22 with or without cosolvents such as ethanol and acetone held under conditions in which the fluids are supercritical, critical or near-critical;
(b) injecting said mixture in a stream into a deareated solution comprising cf one or more sugars in a concentration of 1 to 20%, and a cross-linking agent such a polyvinyl alcohol in a buffer to form one of more spheres having a diameter of 100 to 500 nanometers; and
(c) lyophilizing said one or more spheres to form a lyophilized sphere having a diameter of about 100 to 500 nanometers having a shell comprising hydrophobic polymers containing cannabidiol and naltrexone.

47. The method of claim 46 wherein said hydrophobic polymer is poly (D,L-lactide-coglycolide) in a ratio of 75:25 to 25:75.

48-49. (canceled)

50. The method of claim 46 wherein the poly (D,L-lactide-coglycolide) polymer is pegylated with polyethylene glycol.

51. The method of claim 46 wherein said hydrophobic polymer is selected from the group consisting of polycaprolactone and Eudragit L100.

52. The method of claim 46 wherein said hydrophobic polymer is a mixture selected from the group consisting of poly (D,L-lactide-coglycolide), polycaprolactone, and Eudragit L100.

53-54. (canceled)

55. The method of claim 46 wherein said buffer comprises an alcohol such as ethanol having a concentration ranging from 1 to 50%.

56-59. (canceled)

60. The use of an article comprising a lyophilized sphere having a diameter of about 100 to 500 nanometers having a shell comprising hydrophobic polymers containing cannabinoid and naltrexone for opioid, heroin, morphine and cannabis use disorders; chemotherapeutic induced neuropathic peripheral pain, diabetic neuropathy, peripheral and chronic pain; complex regional pain syndrome; epilepsy and other seizure disorders; multiple sclerosis and dystonia; Parkinson disease; Huntington disease; Alzheimer's disease; Fragile-X syndrome; fibromyalgia; Crohn's disease; graft-versus-host disease (GVHD); cancer; diabetes; anxiety and social anxiety disorder; bipolar disorder; schizophrenia; smoking cessation; insomnia; inflammation; bacterial and viral diseases.

61. (canceled)
